(19) United States
(12) Patent Application Publication
(10) Pub. No.: US 2022/0226553 A1
(43) Pub. Date: Jul. 21, 2022

(54) HEMODIALYSIS ENHANCEMENT APPARATUS & METHOD

(71) Applicant: Global Monitors, Inc., Rancho Santa Fe, CA (US)

(72) Inventor: Jen-Shih LEE, Rancho Santa Fe, CA (US)

(21) Appl. No.: 17/149,793

(22) Filed: Jan. 15, 2021

(51) Int. Cl. A61M 1/30; A61M 1/16

(52) U.S. Cl. CPC A61M 1/308; A61M 1/303; A61M 1/1621; A61M 1/1601

(57) ABSTRACT

A hemodialysis enhancement apparatus involves a bladder having elastically deformable surface that forms a variable volume therewithin. The elastically deformable surface has a smooth interior surface such that blood cannot collect to form a blood clot. The hemodialysis enhancement apparatus further includes a rigid housing having a wall surrounding the bladder and defining a housing volume such that a) when the variable volume chamber has a volume equal to the first volume, most of the elastically deformable surface will be spaced apart from the wall, and b) when the variable volume chamber has a volume equal to the second volume, a substantial portion of the elastically deformable surface will abut the wall. A method performed within a hemodialysis system involves, during an initial phase, withdrawing a volume of a patient's blood into a hemodialysis enhancer, and during a subsequent phase, translocating the patient's blood back into the patient's circulation.

### FIELD OF THE INVENTION

This disclosure relates generally to hemodialysis and, more particularly, to hemodialysis equipment and processes.

### BACKGROUND

Patients having end stage renal disease (“ESRD”) do not have sufficiently functional kidneys to remove extra fluid and metabolic wastes, like accumulated urea, from the body. Annually. roughly 340,000 patients with ESRD undergo hemodialysis (“HD”) treatment, three times a week for 4-5 hours each, in dialysis clinics in the U.S. It is estimated that between 25% and 40% of those patients develop intradialytic hypotension (“IDH”) and/or post dialytic orthostatic hypotension (“PDOH”). IDH can lead to cardiovascular complications. Hypovolemia from either too voluminous or too rapid fluid removal may lead to multiorgan ischemia and associated clinical sequelae. The arterial blood pressure in hemodialysis patients can fluctuate significantly, and high variability in arterial blood pressure is associated with high probability of congestive heart failure and can lead to death.

The KDOQI guidelines of the National Kidney Foundation (National Kidney Foundation, K/DOQI Clinical Practice Guidelines for Cardiovascular Disease in Dialysis Patients, Am. J. Kidney Dis. 45:S1-S154 (2005)) state: “IDH impairs the patient's well-being, can induce cardiac arrhythmias, predisposes to coronary and/or cerebral ischemic events. During the past 10 years, despite improvements in dialysis technology, the frequency of IDH has remained unchanged at about 25% of all HD sessions.”

Attempts have been made to address the IDH problem, but each has undesirable drawbacks.

One approach is to slow down the filtration rate. However, this approach undesirably lengthens the time required for the treatment.

An alternative approach is to simply stop the treatment, send the patient home, and resume treatment the next day. This is expensive, potentially impacts the treatment of other patients, and wastes time.

Another alternative approach that is used when a patient is showing signs of IDH is to infuse saline into the patient's circulation to increase the patient's blood pressure and hence cardiovascular function. However, this saline infusion approach requires that ultrafiltrate, of a similar volume to the saline infused be subsequently extracted, so that the goal set for ultrafiltration can be met at the end of the hemodialysis session.

Another approach involves changing the patient from a supine position to a head down position (the Trendelenburg position) to induce blood to shift from lower body to the central circulation so that cardiac filling can be better maintained. However, it is difficult to use this approach to timely reduce the hemodialysis induced reduction in venous return, cardiac filling, cardiac output and arterial blood pressure.

Yet other approaches involve using sodium modeling, a cooler dialysate, and/or a higher sodium dialysate to reduce hypotensive episodes. Each of these approaches also have undesirable side effects. First many patients can not tolerate the protocol of sodium modeling. Second, cooler temperature dialysis induced shivering and cramping in some patients. Third high sodium dialysate causes excessive thirst in patients.

Still further, while higher ultrafiltration (“UF”) rates are desirable, because they shorten the length of time needed for a hemodialysis session, Assimon M M, Flythe J E, Rapid ultrafiltration rates and outcomes among hemodialysis patients: re-examining the evidence base, Curr Opin Nephrol Hypertens. 24(6): 525-530 (2015) has stated that, with current hemodialysis approaches, compelling observational data demonstrate an association between more rapid UF rates and adverse clinical outcomes.

Thus, there remains a need for hemodialysis systems and approaches that can help avoid IDH without the drawbacks of the foregoing approaches and/or allow for use of higher UF rates.

### SUMMARY

One aspect of this disclosure involves a hemodialysis enhancement apparatus including a bladder having an inlet end, an outlet end, and an elastically deformable surface coupling the first end to the second end so as to form a variable volume therewithin. The variable volume is variable between a first volume and a second volume, where in the second volume is greater than the first volume. The elastically deformable surface has a smooth interior surface between the inlet end and outlet end such that blood, passing from the inlet end, through the variable volume of the bladder, and out the outlet end, cannot collect within the variable volume to form a blood clot. The apparatus further includes a rigid housing having a wall surrounding the bladder and defining a housing volume such that a) when the variable volume chamber has a volume equal to the first volume, most of the elastically deformable surface will be spaced apart from the wall, and b) when the variable volume chamber has a volume equal to the second volume, a substantial portion of the elastically deformable surface will abut the wall.

Another aspect of this disclosure involves a method performed within a hemodialysis system. The method involves, during an initial phase of a hemodialysis session, withdrawing a pre-specified volume of a patient's blood that has exited a dialyzer into a hemodialysis enhancer (“HDE”); and during a subsequent phase of the hemodialysis session, translocating the patient's blood back into the patient's circulation according to a specified protocol such that, at the end of the hemodialysis session, all of the pre-specified volume of patient's blood will have been translocated back into the patient's circulation.

A further aspect of this disclosure involves a method performed within a hemodialysis system. The method involves, during an initial phase of a hemodialysis session, withdrawing a pre-specified volume of a patient's blood that has exited a dialyzer into a hemodialysis enhancer (“HDE”) using a pumping system, operating under control of a computer, that is coupled to the HDE; during a subsequent phase of the hemodialysis session, automatically monitoring the patient's systolic blood pressure using a sensor and receiving a signal from the sensor indicative of the patient's systolic blood pressure; and based upon at least the signal, adjusting the pumping system so as to translocate some of the patient's blood between the HDE and the patient's circulation.

### BRIEF DESCRIPTION OF THE DRAWINGS

This disclosure is further described in the detailed description that follows, with reference to the drawings, in which:

FIG. 1 illustrates, in simplified form, an overview of a conventional hemodialysis system;

FIG. 2 illustrates, in simplified form, an overview of a hemodialysis system made up of at least a hemodialysis enhancer and a pumping system;

FIG. 3 illustrates. in simplified form, an initial state of an example HDE within a dialysis circuit such as shown in FIG. 2;

FIG. 4 illustrates, in simplified form a result, of withdrawal of saline from the cavity of the HDE of FIG. 3;

FIG. 5 is a graph showing one example of three different protocols for operating an HDE;

FIG. 6 is a graph showing the changes in microvascular blood pressure with no dialysis (i.e., ultrafiltration rate set to zero) relative to the corresponding changes if dialysis is performed using an HDE according to each of three protocols;

FIG. 7 is a graph showing the potential change in mean arterial pressure (in mmHg) over time both without and with use of an HDE;

FIG. 8 is a graph similar to that of FIG. 7, except it shows the change in microvascular blood pressure;

FIG. 9 is a graph showing the changes in the volume of fluid restituted from the tissue to the patients circulation (“Jr”) over the course of a hemodialysis session without and with use of an HDE 204; and

FIG. 10 illustrates, in simplified form, a hemodialysis system according to the teachings herein that is the same as FIG. 2 except that it incorporates feedback and an associated protocol.

### DETAILED DESCRIPTION

As described herein, we have devised a system and approach that improves the dialysis process. More particularly, through use of the teachings herein, implementations constructed in accordance therewith can provide one or more of: (1) a way to increase fluid restitution from the tissue to the patient's circulation over the course of hemodialysis treatment, (2) a procedure to reduce the effect of blood volume reduction induced by the ultrafiltration of the dialyzer, (3) a way to shorten the hemodialysis treatment time, (4) a procedure to better manage blood volume reduction, (5) a way to reduce the chance for the patient to develop intradialytic hypotension, and (6) a process to reduce the blood pressure variability of the hemodialysis treatment. As a result, better management of the changes in blood pressure and blood volume of patients undergoing hemodialysis treatment can be achieved, thereby improving hemodialysis care quality.

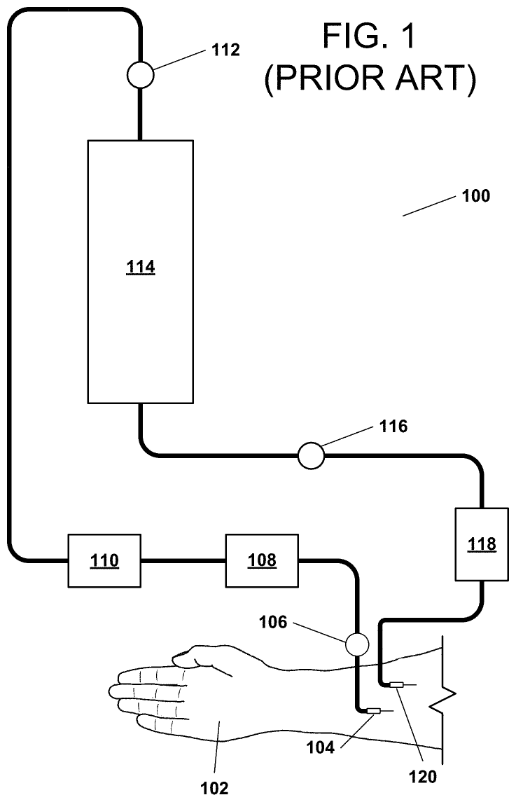

By way of introduction, FIG. 1 illustrates, in simplified form, an overview of a conventional hemodialysis system 100 (a/k/a circuit). In operation, arterial blood flow from a patient 102 is tapped via an arterial catheter 104. The patient's 102 arterial pressure is monitored via an arterial pressure monitor 106 as it is drawn from the patient via a blood pump 108. An anti clotting pump 110 introduces heparin into the blood to help prevent clotting of the patient's blood prior to re-introduction back into the patient 102. A dialyzer pressure monitor 112 is used between the anti-clotting pump 110 and a dialyzer 114 to ensure the blood enters the dialyzer 114 within a proper pressure range. Within the dialyzer 114, the patient's 102 blood gets filtered. Pressure of the patient's blood leaving the dialyzer 114 is monitored via a venous pressure monitor 116 before entering a “device” 118 that acts as an air trap (which may comprise, for example, the air trap, one or more air vent(s) and an air clamp) to prevent any bubbles that may have formed within the blood from entering the user's bloodstream. Finally, the cleaned blood is reintroduced into the patient via a venous catheter 120 inserted into the patient's 102 vein.

In accordance with the teachings herein, we add an additional aspect into the conventional hemodialysis system 100 of FIG. 1 that provides one or more advantages relative to conventional hemodialysis.

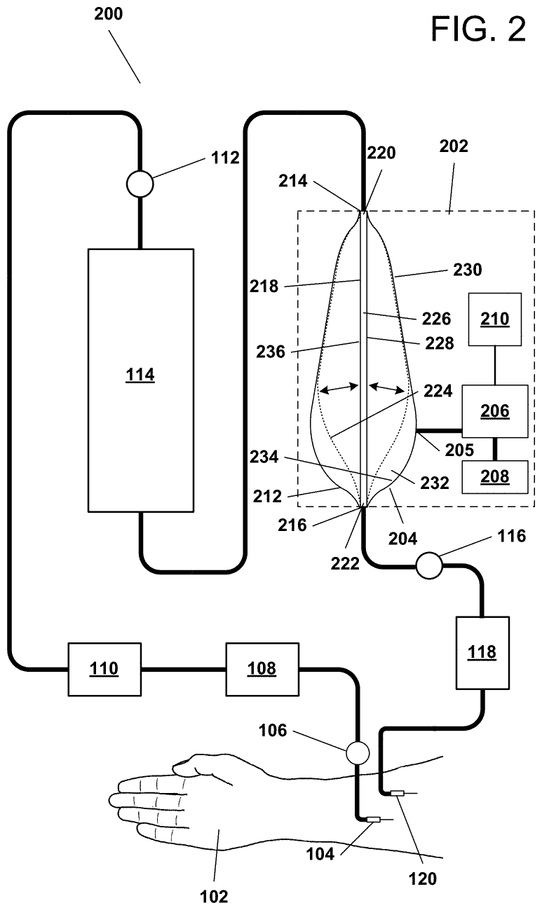

FIG. 2 illustrates, in simplified form, an overview of a hemodialysis system 200 incorporating our additional aspect 202. This aspect 202 is made up of at least a hemodialysis enhancer (“HDE”) 204 and a pumping system 206 that is coupled, via a connection 205, to the HDE 204 such that it can withdraw a fluid (typically, saline) from, and infuse that fluid into, the HDE 204 as described below. The pumping system 206 is coupled to a reservoir 208 for the fluid. In addition, the operation of the pumping system 206 is controlled by a computer 210 operating under program control as described herein. Depending upon the particular implementation, the pumping system 206 can be made up a single, reversible fluid pump, for example a Masterflex peristaltic pump, commercially available from Cole-Parmer, 625 East Bunker Court, Vernon Hills, Ill. 60061. Alternatively, the pumping system 206 can be made up of a pair of independently controllable one way infusion pumps, one whose inlet is connected to the reservoir 208 and whose outlet is connected to the HDE 204 and another infusion pump whose inlet is connected to the HDE 204 and whose outlet connects to the reservoir 208. A further alternative for the pumping system 206 can use a syringe pump, in which case, the interior of the syringe would be the reservoir 208.

HDE 204 Structure

In overview, the HDE 204 is made up of a rigid housing 212 having an inlet 214 coupled to the tubing from the dialyzer 114 and an outlet 216 coupled to the venous pressure monitor 116. A bladder 218 having an inlet 220 and an outlet 222 is constrained within the housing 212 with the bladder inlet 220 coupled to the housing inlet 214 and the bladder outlet 222 coupled to the housing outlet 216 so that the patient's 102 blood from the dialyzer 114 will enter the inlet 220 of the bladder 218, flow through the bladder 218 and out the bladder outlet 222 towards the venous pressure monitor 116. In operation, the HDE 204 will be oriented such that the inlet 214 is above the outlet 216, and ideally directly in a vertical line, although deviations from the vertical that do not affect the operation as described herein, although less desirable, can be acceptable.

The bladder 218 of the HDE 204 is elastically deformable (in a controlled manner as described below) so that it can act like a balloon (i.e., be expanded (as indicated by the arrows and dashed outline) and contracted) such that its interior surface 224 will allow the bladder's 218 interior volume 226 to vary depending upon the extent to which it is expanded/contracted. In general, the bladder 218 is made of a medical grade rubber or polymer that can be expanded to an internal volume of at least 50 times its unexpanded internal volume. Alternatively, the bladder 218 can be made of an electroactive polymer that can comply with the expansion amount and smoothness requirements specified herein. Finally, since the development of new materials is ongoing, it is to be understood that any other material that may exist now or may be developed hereafter that is medically suitable for contact with blood and can satisfy the expansion and smoothness criteria specified herein could be used in accordance with the teachings herein, the particular material selection being largely one of design choice provided the specified criteria can be satisfied.

In implementations where the bladder 218 is made of an electroactive polymer, the pumping system 206 can be replaced by suitable electronic controls that would be actuated by the computer 210.

In general, in its unexpanded state 228, the bladder 218 will appear as a simple tube, whereas, as it expands, for example, to the state 230 where it is at about 80% of the way to the limit of expansion, as shown by the arrows and dashed outline, it will appear more like a balloon. At all degrees from the unexpanded state 228 to the fully expanded state (as constrained by the housing 212), the interior surface 224 is made to be streamlined and smooth (i.e., without surface changes of sufficient degree to provide a place for blood eddies to form downstream of the inlet 212 where blood clots can form).

In addition, a cavity 232 exists between the inner surface 234 (also referred to herein as the wall) of the housing 212 and the outer surface 236 of the bladder 218. In use, changes in the internal volume 226 of the bladder 218 are effected using the pumping system 206 by withdrawing fluid from, or infusing fluid into, the cavity 232. Ideally, initially, the reservoir 208 will contain no fluid and will have a capacity that can contain the maximum fluid that can be received from the cavity 232. In this way, “pinch off” the blood flow within, or collapse of the interior volume 226 of the bladder 218 to less than the unexpanded state 228, cannot occur.

In general, in the unexpanded state 228, the bladder 218 will typically have a volume of between 5 mL and 25 mL, with the inlet and outlet diameters ideally being the same and typically ranging from, depending upon the particular implementation, 0.5 cm to 1 cm. In addition, in general, the housing 212 will have a diameter, at its largest point, measured perpendicular to the unexpanded bladder 218, of 10 cm or less and, most typically, in the range of 8 cm to 5 cm. Still further, some implementations will have multiple size HDEs 204, for example, one with a housing 212 of volume between 150 ml and 175 ml that allows the bladder 218 to expand to have an interior volume 226 of 150 mL or less, another with a housing 212 of volume between 250 ml and 275 ml that allows the bladder 218 to expand to have an interior volume 226 of 250 mL or less, yet another with a housing 212 of volume between 350 ml and 375 ml that allows the bladder 218 to expand to have an interior volume 226 of 350 mL or less. Of course, commercial implementations could involve variations in the sizes of the above, including larger sizes, provided that some aspect is also present to suitably limit direct over withdrawal or over expansion of the bladder 218 such that arterial blood pressure drops by more than 10 mmHg.

At this point it should be noted that the sizing of the housing 212 is ideally designed so that its rigid interior surface 234 will act to physically limit expansion of the bladder 218. In other words, when the bladder 218 is fully expanded, most, if not all, of the outer surface 236 of the bladder 218 will abut the inner surface 234 of the housing 212.

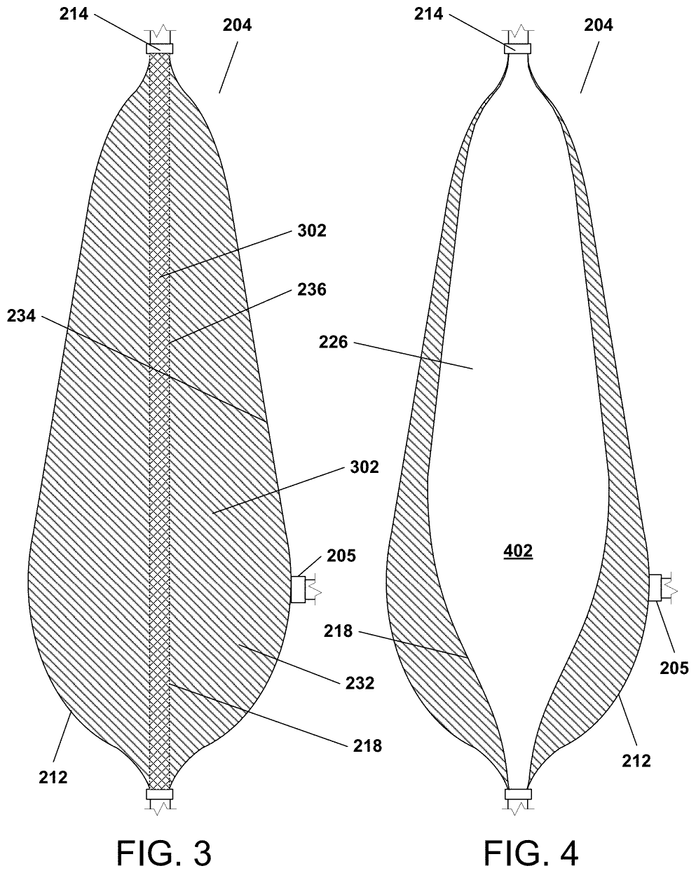

FIG. 3 illustrates. in simplified form, an initial state of an example HDE 204, within a dialysis circuit such as shown in FIG. 2 (but other elements not shown for simplicity), immediately before use, as will be described in greater detail below. As shown, the bladder 218 is in its unexpanded state 228, and is filled with saline 302 (as indicated by the cross hatching). Similarly, the cavity 232 between the inner surface 234 (also referred to herein as the wall) of the housing 212 and the outer surface 236 of the bladder 218 is also filled with a maximum of saline 302 (as indicated by the diagonal hatching) and, as noted above, the reservoir 208 (not shown) is empty. During some initial period thereafter, blood from a patient replaces the saline 302.

FIG. 4 illustrates, in simplified form, a result, of withdrawal of saline 302 from the cavity 232 of the HDE 204 of FIG. 3 into the reservoir 208 by the pumping system 206 so as to cause the bladder 218 to expand, thereby increasing its interior volume 226 and, consequently, the amount of patient blood 402 within the bladder 218. Likewise, infusion of saline 302 back into the cavity 232 from the reservoir 208 by the pumping system 206 will cause the bladder 218 to contract back towards the state shown in FIG. 3.

Finally, with respect to FIGS. 2-4, it should be understood that the specific shape of the housing 212 shown in FIGS. 2-4 is not required, other shapes that are spherical, egg shaped, capsule shaped, bullet shaped, conical, including more complex shapes (such as shown in FIGS. 2-4 or an upside-down version of it), that perform the same limiting and smoothness functions with respect to the bladder 218 can be used. Likewise, instead of using a single HDE 204, some implementations can be created that use two or more smaller HDEs 204 connected to the dialyzer 114 in parallel with each other, or with one connected to the dialyzer 114 and the subsequent HDE(s) 204 connected in series with each other, in either case, with each HDE 204 being independently coupled to a separate pumping system 206 (and, hence, independently controllable), or with all coupled to the same pumping system 206 (such that they operate in concert).

During hemodialysis, advantageously, the HDE 204 can be used to regulate the blood pressure of the patient and thereby reduce or avoid dialysis induced IDH. Various approaches for doing so using an HDE constructed according to the teachings herein will now be described.

Referring back to FIG. 2, to prepare the HDE 204 for use, the HDE 204 is connected into the dialysis circuit through the inlet 220 and outlet 222. At that time, air is typically present inside the bladder 218 and saline is the fluid filling up the cavity 232 between the bladder 218 and the housing 212. Next, the entire hemodialysis circuit is flushed with saline. Then, the ends of the hemodialysis circuit are connected to the arterial and venous catheters 104, 120 that have been respectively inserted to an artery and vein in the patient's 102 arm. Then the blood pump 108 is activated to start the hemodialysis process. Since the bladder 218 is within a saline 302 filled, rigid housing 212, initiation of hemodialysis will not cause any change in the initial bladder 218 interior volume 226. Only, withdrawal or infusion of the saline 302 will effect a change in that interior volume 226. As an aside, it is to be noted that, advantageously, since the bladder 218 is located within a saline 302 filled, rigid housing 212, in the unlikely event of a cracking of the bladder 218 surface, due to the relative pressures within and outside the bladder 218, at worst, minimal temporary exposure of the patient's blood to some saline 302 can occur, which is not hazardous to the patient.

Over the course of hemodialysis, fluid in the blood is usually ultrafiltrated by the dialyzer at a rate of 500 to 1000 mL/hr. This ultrafiltration leads to a 5 to 8 mmHg increase in colloidal osmotic pressure and a 4 to 6 mmHg decrease in the microvascular blood pressure. These two pressure changes cause the rate of fluid restitution to increase from zero to a rate closer to the ultrafiltration rate. The difference between the ultrafiltration rate and restitution rate is the rate change of the total blood volume. An HDE 204 implemented according to the teachings herein, lowers the microvascular blood pressure in the first half of the hemodialysis so that more fluid is restituted from the tissue to counter the effect of ultrafiltration on total blood volume.

As defined by the KDOQI guidelines of the National Kidney Foundation an IDH episode during dialysis is indicated by one of the following two conditions:

(1) A drop in systolic blood pressure of 20 mmHg or more, or

(2) A drop in mean arterial blood pressure of 10 mmHg along with symptoms such as nausea, vomiting and muscle cramping.

To reduce or avoid an IDH episode, in systems employing the teachings herein, if a patient is experiencing or approaching a decrease in systolic blood pressure or mean arterial blood pressure indicative of IDH, the internal volume of an HDE 204 is adjusted to slow down the pressure decrease.

In simplified overview, according to the approaches described herein, soon after the initiation of hemodialysis, some 2 to 5% of the patient's total blood volume is translocated to the HDE 204 over the course of about 10 to 15 minutes, typically over about 0.2 hours (12 minutes). On an individual patient basis, the volume of translocated blood (which is typically calculated as 3% to 5% of the total blood volume based upon the individual's body weight under the assumption that 1 Kg of body weight equates to 80 cc of blood) will not cause the arterial blood pressure to drop by more than 10 mmHg. Moreover, and advantageously, in order to further limit the amount of blood that can be withdrawn to an HDE 204, as noted above, HDEs 204 of different maximum internal volumes 226 can be available as noted above, for example, three different size HDEs 204 with, respectively maximum internal volumes 226 of 150 ml, 250 ml, and 350 ml. In this way, the HDE 204 of 150 ml would be used with patients having a body weight of about 38 Kg or more, the HDE 204 of 250 ml would be used with patients having a body weight of about 63 Kg or more, the HDE 204 of 350 ml would be used with patients having a body weight of about 88 Kg or more. In this way, one can further ensure that no more than the maximum internal volume 226 of an HDE 204 can be withdrawn from the circulation. By way of example, for a patient weighing 72 Kg an HDE 204 of 250 ml would be selected instead of an HDE of 350 ml, so as to ensure that no more than 250 ml could be translocated (i.e., over withdrawal cannot occur).

The blood withdrawn to an HDE 204 during the start of a hemodialysis session is all linearly translocated (i.e., at a constant rate) back to the patient's circulation by the end of the hemodialysis session according to a protocol as will be described below.

Using this process involving an HDE 204, during the first few hours of the hemodialysis, fluid restitution in patients will be larger than without using an HDE 204. This increase in the rate of fluid restitution and the translocation of blood from the bladder 218 reduces the rate of change in blood volume induced by the hemodialysis relative to hemodialysis without the use of an HDE 204. As a result, the chances of the patient developing IDH is reduced or prevented. Moreover, advantageously, the reduction also allows the physician to set the ultrafiltration rate at a higher level without increasing the risk of inducing IDH over the course of hemodialysis. Moreover, if a patient's arterial blood pressure is decreasing rapidly, using an HDE 204, we can translocate blood from it to slow down the pressure drop. Conversely, when the patient's arterial blood pressure shows sign of an adverse increase, blood can be translocated from the patient's circulation to the HDE 204. As a result, undesirable levels of blood pressure variability can be reduced.

With the foregoing overview, a more detailed explanation of example processes for accomplishing the foregoing will now be provided.

For purposes of explanation, the blood volume within the bladder 218 is designated as Vh(t). As noted above, by changing the saline volume within the cavity 232, the HDE withdraws blood from or adds blood to the patient's circulation.

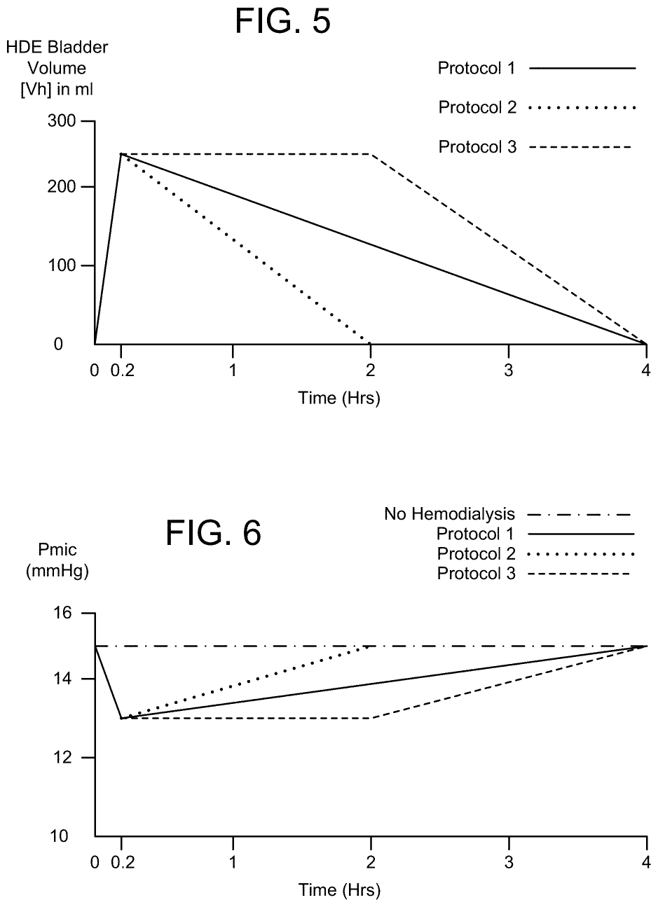

FIG. 5 is a graph showing one example of three different protocols for operating an HDE 204 during a 4-hour dialysis session, where, according to the patient's weight (e.g., 61 Kg) and the formula noted above, 5% of the patient's total blood volume is just under 250 ml.

With continuing reference to FIG. 2 in conjunction with FIG. 5, according to Protocol 1, as shown, by withdrawing saline from the cavity 232 of the HDE 204, the bladder 218 is expanded over the course of about 0.2 hrs so as to retain, within the bladder 218, 250 ml of the patient's circulating blood that has exited the dialyzer 114. As shown, over the course of the remaining time of the session, according to Protocol 1, all blood from the bladder 218 is translocated back to the patient's circulation in a linear manner. On Protocol 2 and 3, the blood is translocated back to the circulation over the first half of the hemodialysis session (the broken line) and the second half of hemodialysis session (the dotted line) respectively.

FIG. 6 is a graph showing the changes in microvascular blood pressure with no dialysis (i.e., ultrafiltration rate set to zero) relative to the corresponding changes if dialysis is performed using an HDE 204 according to Protocol 1, Protocol 2 & Protocol 3.

If the ultrafiltration rate is set as zero, the microvascular blood pressure may be maintained at the constant level of 15 mmHg, as shown in the dash-dot-dash line of FIG. 6. In contrast, with hemodialysis and use of one of Protocol 1, Protocol 2 or Protocol 3, the microvascular blood pressure will initially drop to about 13 mmHg as the HDE 204 bladder 218 fills, but then will return to 15 mmHg in a linear fashion over the dialysis session duration under Protocol 1, will return to 15 mmHg in a linear fashion during the first half of the dialysis session under Protocol 2, and, under Protocol 3, will remain at 13 mm Hg until the start of the second half of the dialysis session before linearly returning to 15 mmHg.

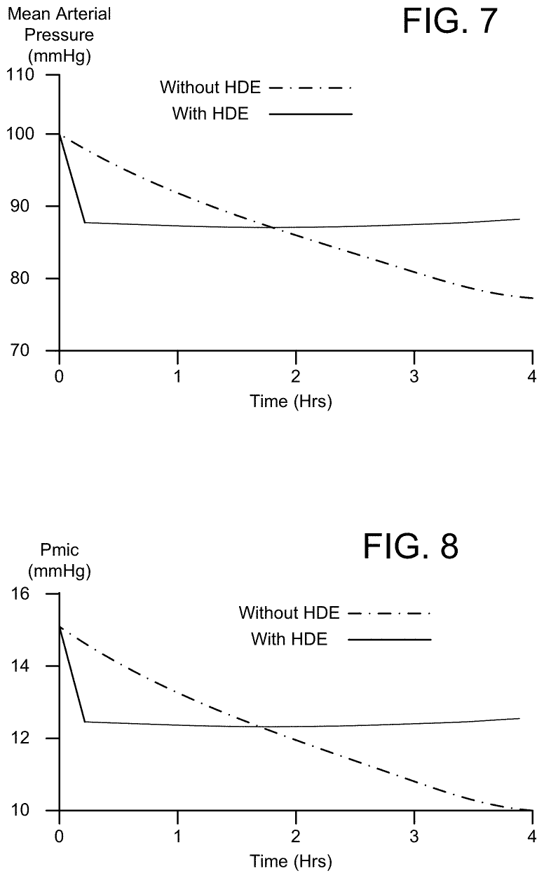

FIG. 7 is a graph showing the potential change in mean arterial pressure (in mmHg) over time both without and with use of an HDE 204. As shown in FIG. 7, normally (i.e., with dialysis performed using a conventional system such as shown in FIG. 1) the continuous fluid extraction by the dialyzer 114, with its the resulting decrease in blood volume, induces a gradual decrease in mean arterial blood pressure (shown by the dash-dot-dash broken line). As shown in FIG. 7, over the course of the session, IDH develops around the 3-hour mark, as the mean arterial pressure drops by more than 20 mmHg from the initial. In contrast, while the use of an HDE 204 will initially lead to a faster reduction in blood volume, and subsequently a drop in mean arterial blood pressure of around 12 mmHg as the bladder 218 is filled to 5% of the patient's total blood volume, thereafter, as more fluid is being restituted from the tissue into the circulation, the mean arterial blood pressure may be maintained at a relatively steady or even slightly increased level. Thus, by using an HDE 204, the mean arterial blood pressure (the solid line) would not drop from its initial value by more than 20 mmHg. As a result, IDH development would be alleviated. Ideally, in actual use, the maximum internal volume set for the bladder 218 should typically be chosen such that the blood volume translocation does not lead to a reduction in mean arterial blood pressure by more than 5 to 10 mmHg. Advantageously, this can be achieved through monitoring of the mean arterial pressure of the patient and feeding the result of that monitoring to the computer 210 such that it halts expansion of the HDE 204 at the point where the reduction in mean arterial blood pressure reaches 10 mmHg.

When the dialyzer 114 is activated to extract fluid at the rate of dVe(t)/dt, a rate of fluid restitution (“Jr”) is induced. As the latter is usually smaller than the former, the total blood volume will be in a decreasing mode, leading to the lowering of the microvascular blood pressure. The rate of fluid restitution, is specified by Starling's principle of transcapillary fluid movement, and is calculated according to Equation 1 as:

Jr=Qlymp+K(−Pmic+πpl+Pt−πt) (Eq. 1)

where “Qlymph” is the lymphatic return, “K” is the filtration coefficient of endothelium membrane, “πpl” is the colloidal osmotic pressure of plasma, “Pt” the interstitial fluid pressure and “πt” is the colloidal osmotic pressure of the interstitial fluid.

FIG. 8 is a graph similar to that of FIG. 7, except it shows the change in microvascular blood pressure both with (shown using a solid line), and without (shown using a broken line), use of an HDE 204. As shown in FIG. 8, a decrease in the microvascular blood pressure will increase the rate of fluid restitution from the tissue into the patient's circulation. As ultrafiltrate is being extracted from the blood passing through the hemodialyzer, the colloidal osmotic pressure will rise over the course of hemodialysis. According to Equation 1, the increase in colloidal osmotic pressure also induces more fluid restitution.

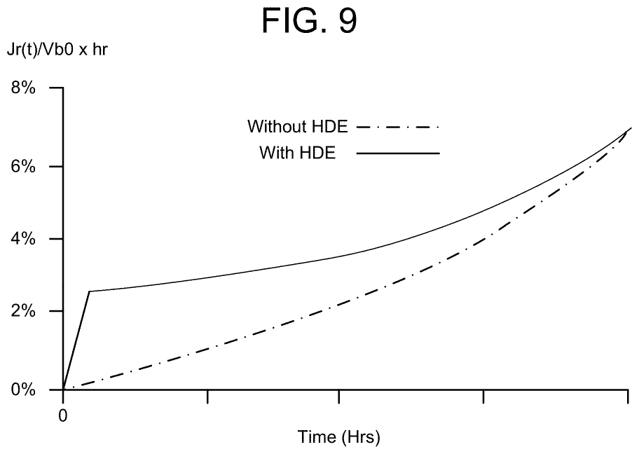

When an HDE 204 is operated under Protocol 1 and assuming that Qlymp, πpl, Pt and πtissue remain unaltered, then the decrease in Pmic (the solid line in FIG. 8) will make Jr have the form depicted as the solid line in FIG. 9.

When the HDE 204 is used, the rate change of blood volume dVb(t)/dt is calculated according to Equation 2 as:

dVb(t)/dt=−dVe(t)/dt+Jr−dVh(t)/dt (Eq. 2)

where Ve(t) is the rate of fluid being ultrafiltrated out of the circulation by the dialyzer 114. If the last term in Equations 2 (dVh(t)/dt) is set to zero, then Equation 2 describes how the blood volume changes without an HDE 204. Over the period from 0.2 hr. to 4 hr. we see that Jr with the HDE 204 activated is larger than that without use of the HDE 204 and that dVh(t)/dt will take a positive value. If the HDE 204 is not activated, then dVh/dt=0. These changes in Jr and dVh/dt are added up through Eq. 2. As a result, the value of dVb/dt with the HDE 204 will be less negative than without use of the HDE 204.

FIG. 9 is a graph showing the changes in Jr over the course of a hemodialysis session without and with use of an HDE 204. The integration of dVb/dt over the dialysis session duration (i.e., from time 0 hours to time 4 hours) yields the net decrease in the total blood volume (Vb4−Vb0). With the changes in Jr shown in FIG. 9, we see that dVb/dt and Vb4−Vb0, with the HDE 204 are less negative than hemodialysis treatment without the HDE 204. The lesser negativity in dVb/dt and Vb4-Vb0 indicates that, through use of the HDE 204, a patient is less likely to develop IDH. Consequently, this allows a physician or appropriate healthcare provider to set the ultrafiltration rate at a higher value than they would without an HDE without risking the patient developing IDH. The use of a higher ultrafiltration rate means that the time to meet the same total ultrafiltration goal of the hemodialysis can be shortened through use of the HDE 204.

It should now be appreciated that the HDE 204 serves as a reservoir for blood from the patient 102 involving translocation of a certain percentage of the total blood volume out of the patient's circulation in the first few minutes of the hemodialysis session followed by controlled translocation of the blood in the HDE 204 slowly back to the patient's circulation. The overall impact of this reinfusion translocation, according to one of the foregoing protocols, is to reduce the rate of blood volume reduction relative to what would occur without use of an HDE 204. This reduction in dVb/dt becomes the key factor in slowing down or preventing development of IDH. It also opens up an avenue for a physician or appropriate healthcare provider to set the ultrafiltration rate to a higher value, shortening the hemodialysis time while still meeting the goal set for the ultrafiltration.

In use, hemodialysis patients are be classified into one of three groups:

Group 1: Patients that have had no IDH episodes over their hemodialysis treatment in the prior two months or those who have had an IDH episodes at different times within the hemodialysis period. For those patients, Protocol 1 is employed so that, over the period following expansion of the bladder 218 to the specified maximum for that patient based upon their weight as described above, (i.e., 0.2 hr to 4 hr), the HDE 204 translocates blood from the bladder 218 to the patient's circulation through infusion of saline from the reservoir 208 by the pumping system 206 into the cavity 232 to reduce the interior volume 226 of the bladder 218 and thereby generate a positive dVh/dt to reduce the rate decrease in total blood volume (dVb/dt). The reduced rate of decrease slows down or prevents the patient from developing IDH that can otherwise occur over the course of a hemodialysis treatment session.

Group 2: Patients that have had IDH episodes mostly in the first half of their hemodialysis sessions. For those patients, Protocol 2 is employed because it is designed to provide a positive dVh/dt to lower the rate of volume decrease during that first half and, hence, the likelihood for the patient to develop IDH in the first half.

Group 3. Patients have had IDH episodes mostly in the second half of their hemodialysis sessions. For those patients, Protocol 3 is employed to generate a positive dVh/dt during that second half of the hemodialysis session to make dVb/dt less negative and, hence, the patient's development of IDH less likely.

Optionally a fourth group and protocol can be defined in which the HDE 204 is automatically operated through feedback.

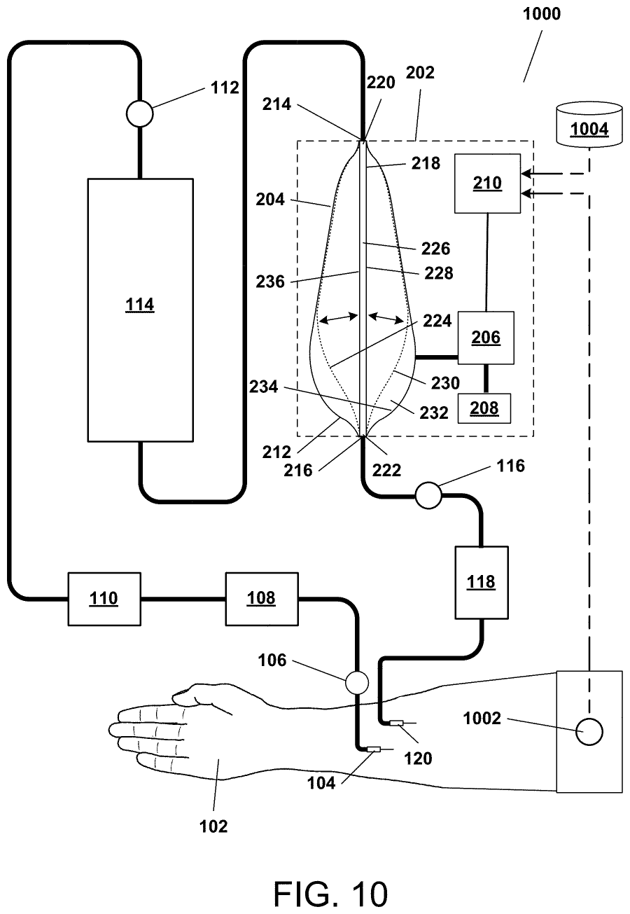

FIG. 10 illustrates, in simplified form, a hemodialysis system 1000 according to the teachings herein that is the same as FIG. 2 except that it incorporates feedback and an associated protocol.

Specifically, as shown, the system 1000 further includes a sensor 1002, coupled to the patient 102, that measures the patient's 102 systolic blood pressure, and non-transitory storage 1004 to which the computer 210 has access (wired or wireless, depending upon implementation).

During a dialysis session, the patient's 102 systolic blood pressure measurements are fed back in a signal to the computer 210, through a wired or wireless connection. The computer 210 monitors the signal from the sensor 1002 on, depending upon the particular implementation, a periodic or asynchronous basis. The received systolic blood pressure measurements are, again depending upon the particular implementation, (i) stored locally in RAM or non-transitory storage for the duration of the hemodialysis session and written into other non-transitory storage 1004 following completion of the hemodialysis session, (ii) written to the non-transitory storage 1004 shortly after receipt, or (iii) accumulated and written to the non-transitory storage 1004 on a specified schedule.

In addition, the computer 210 has access to systolic blood pressure readings from the patient's 102 past hemodialysis treatments.

According to this fourth protocol, a patient's systolic blood pressure is, depending upon the particular implementation, periodically (e.g., every 15 minutes) or continuously measured. A baseline blood pressure reading is taken at the start of the hemodialysis session. Then, after the initial blood volume translocation to the HDE's 204 bladder 218 and receipt by the computer 210 of a subsequent systolic blood pressure reading from the sensor 1002, the computer 210 will analyze the systolic blood pressure reading and pressure readings from the patient's 102 past hemodialysis treatments obtained from the storage 1004 and, based thereon, send an instruction to the pumping system 206 to translocate blood in the HDE 204 back to the patient's circulation at a designated linear rate over the next 15 minutes. The process of calculation and adjusting of the pumping system 206 are continued through the hemodialysis, with the programming being configured such that, irrespective of the intervening adjustments to the HDE 204 the bladder 218 will be returned to its unexpanded state at the end of the hemodialysis treatment session.

For this protocol, the computer would be programmed to recognize whether the blood pressure is decreasing at a rate higher than normal and, if so, set the blood translation from the bladder 218 to the patient's 102 circulation at a higher rate to counter the development of hypotension. Similarly, if the patient's 102 systolic blood pressure readings received from the sensor 1002 shows signs of wide fluctuations, the computer 210 will vary the blood volume within the bladder 218 so as to reduce the pressure fluctuation. Advantageously, as a result, through this feedback protocol undue adverse blood pressure variability during the hemodialysis session can be reduced or eliminated.

It should now be appreciated that, implementations employing the teachings herein can address the drawbacks noted above.

Specifically, through use of some implementations employing the teachings herein, the ultrafiltration rate can advantageously be sped up, reducing the time required for reaching the same goal of a hemodialysis session relative to the time for a conventional hemodialysis session.

Most implementations employing the teachings herein can eliminate the need to stop and reschedule hemodialysis session due to the patient developing IDH, because the implementations are specifically directed to avoiding an IDH episode in the first instance.

Implementations employing the teachings herein, provide distinct advantages over a conventional saline infusion approach which infuses saline into a patient's the circulation to improve venous return and hence cardiovascular function by increasing the patient's blood pressure to counter or slow down the developing IDH. Advantageously, through use of an HDE according to the teachings herein, the patient's blood is translocated back into their circulation to slow down the decrease in blood pressure. Thus, since implementations employing the teachings herein do not involve saline being added to the patient's circulation, additional ultrafiltrate extraction is unnecessary.

Still further, by employing the teachings herein, cardiac filling can be more reliably maintained relative to shifting the patient into a Trendelenburg position.

Finally, implementations employing the teachings herein have advantages over approaches involving using sodium modeling, a cooler dialysate, and/or a higher sodium dialysate. First, the implementations employing the teachings herein avoid the need to resort to a sodium modeling protocol. Second, the implementations employing the teachings herein can operate at the temperature normally employed in a hemodialysis session, and thereby avoid the need to resort to cooler temperature dialysis. Third, the implementations employing the teachings herein avoid the need to resort to use of high sodium dialysate.

Having described and illustrated the principles of this application by reference to one or more examples, it should be apparent that embodiment(s) may be constructed and/or modified in arrangement and detail without departing from the principles disclosed herein and that it is intended that the application be construed as including all such modifications and variations insofar as they come within the spirit and scope of the subject matter disclosed.

The foregoing outlines, generally, the features and technical advantages of one or more implementations that can be constructed based upon the teachings in this disclosure in order that the following detailed description may be better understood. However, the advantages and features described herein are only a few of the many advantages and features available from representative examples of possible variant implementations and are presented only to assist in understanding. It should be understood that they are not to be considered limitations on the invention as defined by the appended claims, or limitations on equivalents to the claims. For instance, some of the advantages or aspects of different variants are mutually contradictory, in that they cannot be simultaneously present in a single embodiment. Similarly, some features or advantages may be applicable to one aspect and inapplicable to others. Thus, the foregoing features and advantages should not be considered dispositive in determining equivalence. Additional features and advantages will be apparent from the teachings of the description, drawings, and claims.

1-14. (canceled)

15. A method performed within a dual catheter hemodialysis system, comprising:
during an initial phase of a hemodialysis session for a patient in which the patient's blood is withdrawn from the patient via a first catheter, exits a dialyzer into a hemodialysis enhancer (“HDE”) and returns to the patient via a second catheter, reducing the patient's blood volume by expanding an internal volume of an elastically deformable bladder of the HDE to a pre-specified volume, wherein the bladder includes an inlet and an outlet through which the patient's blood will continuously flow during the hemodialysis session, the bladder being positioned within a rigid chamber, and the bladder including a smooth interior surface between the inlet and the outlet the patient's blood, flowing through the inlet, the bladder and out the outlet, cannot collect within the bladder to form a blood clot; and
during a subsequent phase of the hemodialysis session, increasing the patient's blood volume by decreasing the internal volume of the bladder according to a specified protocol, and wherein, at an end of the hemodialysis session, all of the patient's blood in the dialyzer and bladder will have been returned to the patient's circulation via the second catheter.

16. The method of claim 15, wherein the subsequent phase ends halfway through the hemodialysis session.

17. The method of claim 15, wherein the subsequent phase begins halfway through the hemodialysis session.

18. The method of claim 15, wherein the returning of the patient's blood to the patient's circulation is done at a constant rate.

19. A method performed within a dual catheter hemodialysis system comprising:
during an initial phase of a hemodialysis session for a patient in which the patient's blood is withdrawn from the patient via a first catheter, exits a dialyzer into a hemodialysis enhancer (“HDE”) and returns to the patient via a second catheter, reducing the patient's blood volume by expanding an internal volume of an elastically deformable bladder of the HDE to a pre-specified volume, wherein the bladder includes an inlet and an outlet through which the patient's blood will continuously flow during the hemodialysis session, the bladder including a smooth interior surface between the inlet and the outlet such that the patient's blood, flowing through the inlet, the bladder and out the outlet, cannot collect within the bladder to form a blood clot, wherein a pumping system, operating under control of a computer, is coupled to and acts upon an exterior surface of the bladder to change the bladder's internal volume;
during a subsequent phase of the hemodialysis session, automatically monitoring the patient's systolic blood pressure using a sensor and receiving a signal from the sensor indicative of the patient's systolic blood pressure; and
based upon at least the signal, adjusting the pumping system so as to alter the interior volume of the bladder and thereby change the blood volume of the patient during the subsequent phase of the hemodialysis session.

20. The method of claim 19, wherein during the subsequent phase, the pumping system is adjusted, under the control of the computer, such that, at an end of the hemodialysis session, all of the patient's blood in the dialyzer and bladder will have been returned to the patient's circulation via the second catheter.
